(19) United States
(12) Patent Application Publication
(10) Pub. No.: US 2005/0038425 A1
(43) Pub. Date: Feb. 17, 2005

(54) Device for externally immobilizing broken bones

(76) Inventors: Gerd Werding, Ingolstadt (DE); Willi Schneider, Bad Neustadt a.d. Saale (DE)

Correspondence Address: NIXON PEABODY, LLP, 401 9TH STREET, NW, SUITE 900, WASHINGTON, DC, 20004-2128, US

(21) Appl. No.: 10/504,090

(22) PCT Filed: Feb. 10, 2003
(86) PCT No.: PCT/EP03/01295

(30) Foreign Application Priority Data
Feb. 11, 2002 (DE) 202 02 048.7

(51) Int. Cl. A61B 17/58

(52) U.S. Cl. 606/54

(57) ABSTRACT

The invention relates to a device for externally immobilizing broken bones, particularly bones of the extremities. According to the invention, interspaced bone pins (9), which are provided for insertion into the bone while passing through the skin and soft-tissue mantle are arranged on a multi-articulation rod. This multi-articulation rod can be placed on the outside of the body while extending along a broken bone and comprises elements (1), which are interconnected in a ball-and-socket type manner and which can be fixed in selected relative positions. The elements (1) have a ball (2) on one end and a ball socket (3) on the other end, and the ball (2) of one element is located inside the ball socket (3) of an adjacent element. The ball and ball socket each geometrically extend over more than one hemisphere, and the surfaces of the ball (2) and ball socket (3) are roughened.

The invention concerns a device for externally immobilizing broken bones as set out in the preamble of claim 1.

DE 19746687 A1 discloses a device of this type. This known device is disadvantageous in that the ball and ball sockets of the ball-and-socket joints are made with smooth surfaces, with the result that excessively strong fastening forces must be used to fix the elements in their relative positions, if the immobilizing device is to be truly rigid even when under stress.

Therefore the problem that this invention addresses is to improve a device of the type indicated above in such a way that it will have greater form stability in its secured state.

This problem is solved in accordance with the invention by a device for externally immobilizing broken bones with the distinguishing features of claim 1.

Advantageous further developments of the invention are the object of the other claims.

The following describes the embodiment of the invention illustrated in the attached drawings. In the drawings:

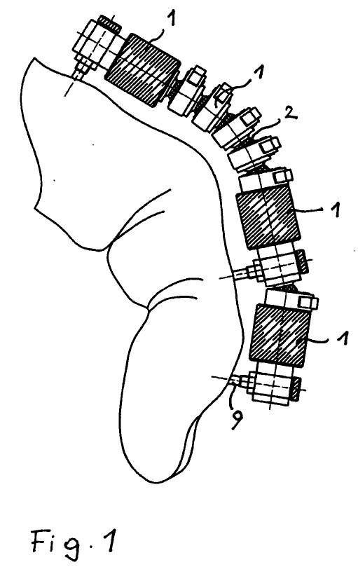

FIG. 1 illustrates the immobilization device according to the invention used on a finger,

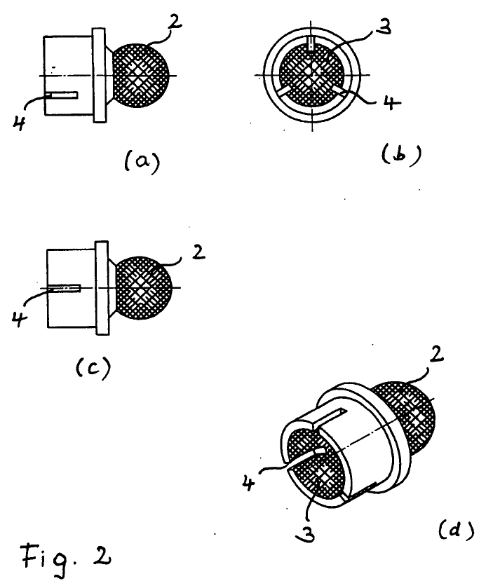

FIG. 2(a)-(d) illustrate an element of the immobilization device according to the invention showing a side view, a rear view, a view from above and a view in perspective, and

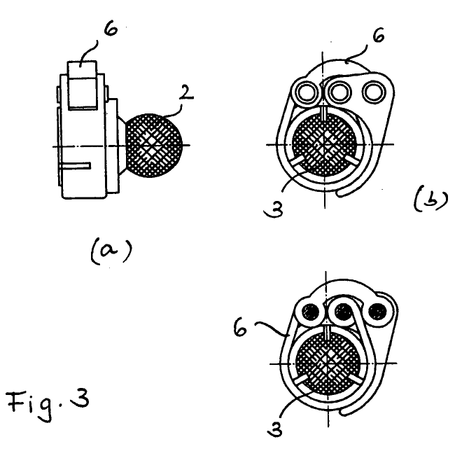

FIG. 3(a)-(c) illustrate the element in FIG. 2 with a mounted clamp showing a side view, rear view and a view from above with the clamp partially removed.

FIG. 1 illustrates the device for externally immobilizing broken bones used on a finger. The device features a multi-articulation rod with elements 1 as they are shown in FIGS. 2 and 3. In appropriate places, the simple elements 1 are replaced with the type of element that holds a bone pin 9.

Elements 1, with the exception of the terminal elements, are provided with a ball 2 at one end and a ball socket 3 at the other end. The ball socket 3 extends geometrically over more than one hemisphere of a ball, preventing joined elements from accidentally detaching from each other. The elements are joined through an elastic deformation of the ball socket that is relieved through slits 4. Neighbouring elements 1 are fixed in their relative positions with the help of the clamp 6 enclosing the ball sockets, by means of which ball socket 3 in tightened state is pressed against the ball 2 of the adjacent element that is contained by that ball socket.

To improve the immobilization of the neighbouring elements relative to one another through the even pressure of clamp 6, the surfaces of ball 2 and ball socket 3 are roughened.

The surfaces of metal parts, and therefore of a ball and ball socket made of metal, can suitably be roughened through spark erosion. In the case of balls and ball sockets made of plastic, a similar surface structure can be achieved by spark eroding the surface of the moulds in which the parts are formed, in particular those parts that determine the form of the ball and ball sockets. The rough surface of the moulds achieved in this way will then be replicated in the products issuing from the moulds.

Another way of obtaining surface roughness consists of milling the ball and ball socket.

In the case of plastics, a rough coating on top of the originally smooth surfaces of the ball and ball socket can also be used. Another method with a view of improving the stability of the relative position of adjacent attached elements consists of forming Velcro-type hook and loop surfaces on the balls and ball sockets.

1. A device for externally immobilizing broken bones, particularly bones of the extremities, in which spaced bone pins (9) which are provided for insertion into the bone through the skin and soft tissue are arranged on a multi-articulation rod which can be placed on the outside of the body extending along a broken bone, said rod comprising elements (1) which are interconnected in a ball-and-socket type manner and which can be fixed in selected relative positions, elements (1) having a ball (2) on one end and a ball socket (3) on the other end, the ball (2) of one element being located inside the ball socket (3) of an adjacent element, characterised in that the ball and ball socket each geometrically extend over more than one hemisphere, and the surface of the ball (2) and ball socket (3) are roughened.

2. A device as claimed in claim 1, characterized in that the ball (2) and ball socket (3) have metal surfaces, and the surfaces have been spark eroded.

3. A device as claimed in claim 1, characterized in that the ball (2) and ball socket (3) are made of plastic that has been formed in moulds with spark-eroded surfaces.

4. A device as claimed in claim 1, characterized in that the balls (2) and ball sockets (3) have Velcro-type hook and loop surfaces.
